(19) United States
(12) Patent Application Publication
(10) Pub. No.: US 2021/0196485 A1
(43) Pub. Date: Jul. 1, 2021

(54) Mechanical Parts Including Pyrolytic Carbon Brace Members

(71) Applicant: Jordan Townsend, Portland, OR (US)

(72) Inventor: Jordan Townsend, Portland, OR (US)

(21) Appl. No.: 16/729,382

(22) Filed: Dec. 28, 2019

(51) Int. Cl. A61F 2/856; A61L 31/02; A61F 2/90

(52) U.S. Cl. CPC A61F 2/856; A61L 2400/12; A61F 2/90; A61L 31/024

(57) ABSTRACT

The present disclosure is directed to mechanical parts including a brace member. The brace member includes a first carbon fullerene and a second carbon fullerene. The first carbon fullerene forms a substantially spherical structure and is pyrolyzed. The second carbon fullerene is contained within the substantially spherical structure of the first carbon fullerene. In some examples, the mechanical parts define a stent. The stent includes an outer tube, an inner tube, and a brace member. The inner tube is coaxially aligned with the outer tube and contained within the outer tube. The brace member of the stent is disposed between the inner tube and the outer tube and is comprised of pyrolytic carbon.

### BACKGROUND

The present disclosure relates generally to mechanical parts. In particular, mechanical parts including pyrolytic carbon brace members are described.

Mechanical parts are utilized in a wide variety of fields. One particular field where mechanical parts find application is nanoscale mechanical parts. Medical devices are one important category of devices making use of nanoscale mechanical parts. Stent for various uses inside the body are an interesting and lifesaving category of medical devices utilizing nanoscale mechanical parts.

Known stents are not entirely satisfactory for the range of applications in which they are employed. For example, existing stents can not be readily created in variety of different configurations. Further, conventional stents are generally specific to a given body part, such as a vein or a heart chamber, and can not be adapted to be used in other parts of the body, such as a kidney or other areas of the body that would benefit from being separated.

In addition, currently known stents do not enable access holes to be formed wherever needed on the stent. Forming access holes allows the stent to remain in place in the body when a surgeon needs to access areas inside the stent. In contrast, conventional stents without suitable access holes must generally be removed when a doctor needs to access regions blocked by the stent.

Another drawback of conventional stents is their relatively poor combination of flexibility and strength, which limits their application in a person's body. Similarly, currently known stents can not be satisfactorily formed with desired levels of flexibility and/or strength. Stents currently available are also not as strong as would be desired.

Current stents are undesirably thick and bulky, which can limit blood flow. Clots are also more prone to form in thicker and bulkier stents.

Conventional stents further lack features to help secure the stent in a desired position. It would be desirable to have a strong, thin, flexible stent that could anchor itself in the body rather than being prone to moving around without external means to secure the stent in position.

Thus, there exists a need for stents that improve upon and advance the design of known stents. Examples of new and useful stents relevant to the needs existing in the field are discussed below.

Disclosure addressing one or more of the identified existing needs is provided in the detailed description below. Examples of references relevant to stents include U.S. Patent Application Publication 2008/0200976 A1. The complete disclosure of the above patent application is herein incorporated by reference for all purposes.

### SUMMARY

The present disclosure is directed to mechanical parts including a brace member. The brace member includes a first carbon fullerene and a second carbon fullerene. The first carbon fullerene forms a substantially spherical structure and is pyrolyzed. The second carbon fullerene is contained within the substantially spherical structure of the first carbon fullerene. In some examples, the mechanical parts define a stent. The stent includes an outer tube, an inner tube, and a brace member. The inner tube is coaxially aligned with the outer tube and contained within the outer tube. The brace member of the stent is disposed between the inner tube and the outer tube and is comprised of pyrolytic carbon.

### BRIEF DESCRIPTION OF THE DRAWINGS

FIG. 1 is a schematic cross section view representing a simplified section of a first stent embodiment perpendicular to longitudinal axis of the stent, the stent depicted in FIG. 1 including different types of pyrolytic carbon brace members extending between an inner wall and an outer wall of the stent.

FIG. 2 is a schematic cross section view representing a simplified section of a second stent embodiment perpendicular to the longitudinal axis of the stent with all pure pyrolytic carbon atoms depicted as circles. FIG. 2 depicts a stent including different types of pyrolytic carbon brace members between an inner wall and an outer wall, holes formed in the inner and outer walls of the stent to provide bonding sites for the brace members, and lines depicting bonds between the brace members and the inner and outer walls at the holes.

FIG. 3 is a perspective view of a third stent embodiment with brace members comprised of carbon fullerenes between an inner wall and an outer wall, the outer wall having terminal ends that flare outwards.

FIG. 4 is a cross section view of a section of a fourth stent embodiment perpendicular to a longitudinal axis of the stent.

FIG. 5 is top plan view of the fourth stent embodiment, the fourth stent embodiment including a higher concentration of brace members than are depicted in the stent example shown in FIG. 3 to yield a stiffer stent.

FIG. 6 is a close up view of a portion of the fourth stent embodiment depicting the arrangement of brace members and chemical bonds between different atoms and molecules.

### DETAILED DESCRIPTION

The disclosed mechanical parts will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, examples of various mechanical parts are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

### Mechanical Parts Including Pyrolytic Brace Members

With reference to the figures, mechanical parts including pyrolytic brace members will now be described. The mechanical parts discussed herein function to provide mechanical structures with advantageous strength and flexibility properties. In some examples, the mechanical parts define stents used in medical applications inside the body. The discussion below will focus on stents, but the reader should understand that the mechanical parts may define a wide variety of other devices in addition or alternatively to stents.

The reader will appreciate from the figures and description below that the presently disclosed stents address many of the shortcomings of conventional stents. For example, the stents described below can be readily created in a variety of different configurations and are not limited to a given body part. The ability to readily modify how the stents are formed allows them to be used in a wide range of body parts, such as a vein, an artery, a heart chamber, a kidney, or in other areas of the body that would benefit from being separated.

An improvement over conventional stents is the fact that the stents described herein enable access holes to be formed wherever needed on the stent. Forming access holes allows the presently described stents to remain in place in the body when a surgeon needs to access areas inside the stent. The access holes are a significant improvement over conventional stents, which lack suitable access holes and must generally be removed when a doctor needs to access regions blocked by the stent.

A benefit of the stents described in this document over conventional stents is their favorable combination of flexibility and strength and their ability to be formed with desired combinations of flexibility and strength. Whereas conventional stents have limited application in a person's body due to being too rigid or not strong enough, the stents described herein can be used effectively throughout the body.

Unlike current stents, which are undesirably thick and bulky, the stents described in this document are relatively thin and compact. The thin design of the presently described stents helps promote increased blood flow through the stent and reduces the likelihood of clots forming.

Another advantage of the stents described herein is their features to help secure the stent in a desired position. The presently described stents are strong, thin, and flexible and include features to help anchor the stents in the body. The stent's ability to anchor itself overcomes the problem of conventional stents being prone to moving around absent external means to secure the stent in position.

### Mechanical Part in the Form of a Stent Embodiment One

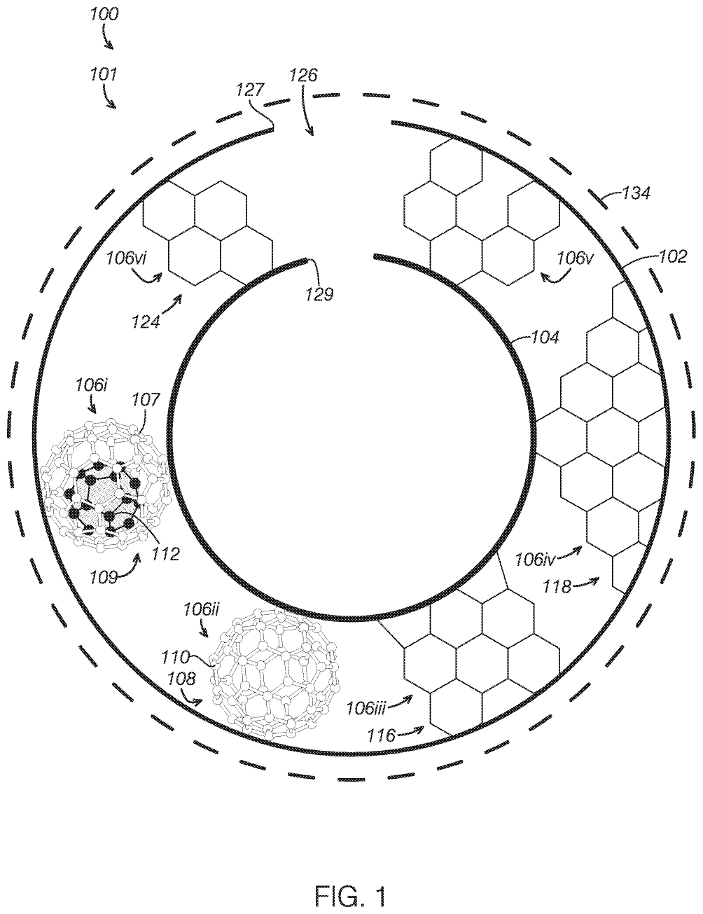

With reference to FIG. 1, a first example of a mechanical part, mechanical part 101, will now be described. Mechanical part 101 defines a stent 100 and includes a first wall 130, a second wall 132, and a plurality of brace members 128. Expressed another way, first wall 130, second wall 132, and plurality of brace members 128 collectively define stent 100. In some examples, the mechanical part includes a single brace member or a small number of brace members rather than a plurality of brace members.

Walls

In the present example, first wall 130 forms an inner tube 104 and second wall 132 forms an outer tube 102. In other examples, one or more of the first and second walls are not formed into tubes, but instead define other configurations. For example, one or more of the walls may define sheets or other configurations with planar portions.

As can be seen in FIG. 1, inner tube 104 and outer tube 102 have circular cross sections. In other examples, the cross sections of the tubes are oval, triangular, rectilinear, a regular polygon, or an irregular shape.

As shown in FIG. 1, inner tube 104 is contained within outer tube 102. In the present example, inner tube 104 and outer tube 102 are coaxially aligned. In other examples, the tubes have longitudinal axes that are not commonly aligned.

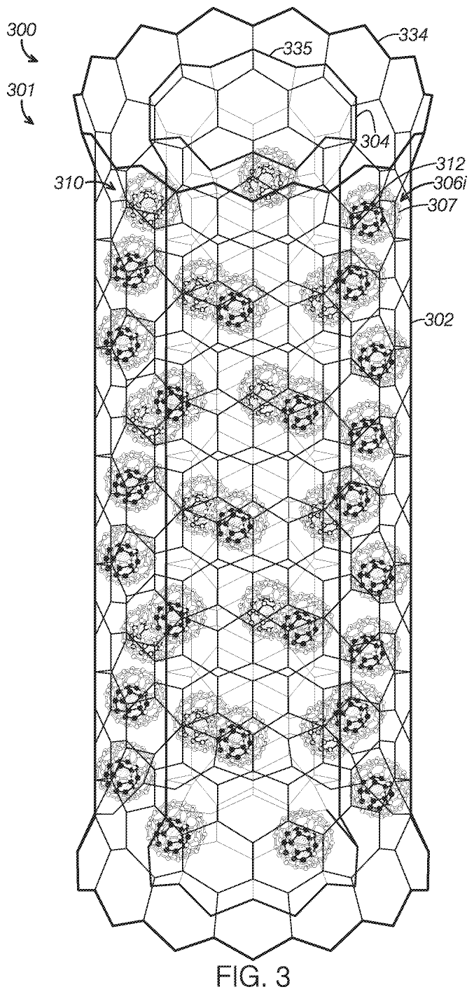

As shown in FIG. 1 in dashed lines, outer tube 102 flares at a terminal end 134 to help anchor stent 100 in a desired position. While not depicted in FIG. 1, the inner tube may also flare outwards, such as shown in FIG. 3 with an inner tube 304 flaring outwards at a terminal end 335. The ends flaring outwards helps the stent lodge against surrounding tissue to hold the stent in place. Additionally or alternatively, the outer wall may define projections and/or peaks and valleys to lodge against surrounding tissue to help hold the stent in place.

In the example shown in FIG. 1, inner tube 104 and outer tube 102 are comprised of carbon nanotube. However, the inner tube and outer tube may be comprised of any currently known or later developed material suitable for use in a given application. In some examples, the inner tube is comprised of a different material than the outer tube.

In the example shown in FIG. 1, outer tube 102 and inner tube 104 collectively define a port 126 extending from outer tube 102 to inner tube 104. Port 126 provides access to an interior of inner tube 104 through a first hole 127 formed in outer tube 102 and a second hole 129 formed in inner tube 104. One may use port 126 to access fluid within inner tube 104 or to dispense medications within inner tube 104 among other uses.

As shown in FIG. 1, brace member 106 is proximate port 126 defined by outer tube 102 and inner tube 104. Brace member 106 being disposed proximate port 126 provides structural stability for port 126.

Brace Members

In the example shown in FIG. 1, plurality of brace members 128 includes six brace members 106i-106vi (collectively 106) in the section of stent 100 depicted. Brace members 106 are disposed between inner tube 104 and outer tube 102. Each brace member may selectively be or not be chemically bonded to one or both of the inner tube and the outer tube. In certain examples, the plurality of brace members includes less than six brace members while other examples include more than six brace members. Some examples include a single brace member.

The number of brace members may be selected to provide a desired amount of stiffness between outer tube 102 and inner tube 104. In general, the more brace members are present the stiffer the stent will be. The number of brace members may also take into account the number of ports formed in the stent with additional brace members being provided proximate the ports to provide mechanical support to the ports.

As can be seen in FIG. 1, plurality of brace members 128 includes a variety of different brace members, namely brace members 106i-106vi. In particular, plurality of brace members 128 includes six different types of brace members 106. Other stent examples include a single type of brace member, a couple different types of brace members, a variety of brace members of less than six different types, or more than six different types of brace members. The different types of brace members may be selected for the mechanical or chemical properties they provide and/or for facilitating manufacturing of the mechanical part.

Brace members 106 are comprised of pyrolytic carbon in various configurations. Pyrolytic carbon is carbon material in a given configuration that has undergone pyrolysis. Pyrolysis involves thermally decomposing carbon material at elevated temperatures in an inert atmosphere to change the chemical composition of the carbon material.

With reference to FIG. 1, the reader can see that brace member 106i includes a pyrolyzed carbon fullerene 107 forming a substantially spherical structure 109. Brace member 106i further includes a second carbon fullerene 112 contained within substantially spherical structure 109 of carbon fullerene 107. Second carbon fullerene 112 is not pyrolyzed, which facilitates it bonding to pyrolyzed carbon fullerene 107 at a desired position inside substantially spherical structure 109.

To insert the inner spherical fullerene in the outer spherical fullerene, a stepwise process may be employed. In a first step, half of the outer spherical fullerene may be formed. In a second step, the inner spherical fullerene may be chemically bonded the outer spherical half at a desired position. Then a second half of the outer spherical fullerene may be formed and added to the first half of the outer spherical fullerene to enclose the inner spherical fullerene.

In the present example, pyrolyzed carbon fullerene 107 comprises C₆₀ carbon and second carbon fullerene 112 comprises Cm carbon. In other examples, different numbers of carbon atoms are present in the respective fullerenes. Second carbon fullerene 112 is chemically bonded to pyrolyzed carbon fullerene 107 within substantially spherical structure 109 of pyrolyzed carbon fullerene 107.

As can be seen in FIG. 1, brace member 106ii includes a pyrolyzed carbon fullerene 110 defining a substantially spherical structure 108. However, as shown in FIG. 1, pyrolyzed carbon fullerene 110 does not include an interior fullerene like second carbon fullerene 112 inside pyrolyzed carbon fullerene 107.

With continued reference to FIG. 1, brace member 106iii includes pyrolytic carbon forming a sheet 116 and brace member 106iv includes pyrolytic carbon forming a pyramid 118. The reader can further see in FIG. 1 that brace member 106v includes super pyrolytic carbon and that brace member 106vi includes pyrolytic carbon interconnected with non-pyrolytic carbon in a zig-zag arrangement 124. A wide variety of other configurations are contemplated, and the brace members may adopt any currently known or later developed arrangement of pyrolytic carbon suitable for a given application.

### Additional Embodiments

With reference to the figures not yet discussed, the discussion will now focus on additional mechanical part embodiments in the form of stents. The additional embodiments include many similar or identical features to stent 100. Thus, for the sake of brevity, each feature of the additional embodiments below will not be redundantly explained. Rather, key distinctions between the additional embodiments and stent 100 will be described in detail and the reader should reference the discussion above for features substantially similar between the different stent examples.

### Mechanical Part in the Form of a Stent Embodiment Two

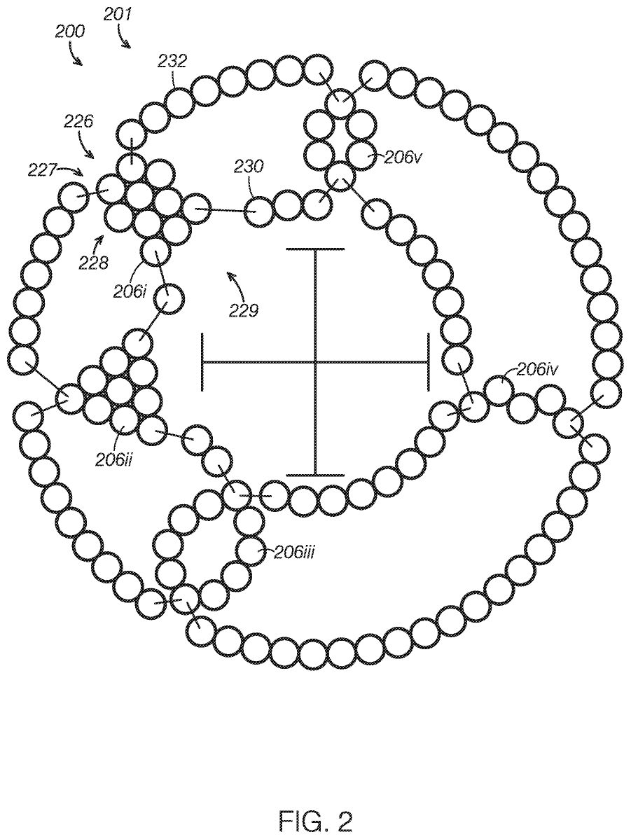

Turning attention to FIG. 2, a second example of a mechanical part in the form of a stent, mechanical part 201 in the form of a stent 200, will now be described. In FIG. 2, the circles depict pure pyrolytic carbon atoms and the central crossed lines depict a flow channel. As can be seen in FIG. 2, stent 200 includes a first wall 230, a second wall 232, and a plurality of brace members 228. First and second walls define inner and outer tubes, respectively.

The reader can see in FIG. 2 that stent 200 includes different types of pyrolytic carbon brace members 206i-206v between first wall 230 and outer wall 232. Brace member 206i defines a sheet of pyrolytic carbon atoms. Brace member 206ii defines a pyramid of pyrolytic carbon atoms, which is a three dimensional structure effective to absorb angular forces. Brace member 206iii depicts C₆₀ fullerene. Brace member 206iv defines pyrolytic carbon in a zig-zag arrangement. Brace member 206v is comprised of extremely pyrolyzed, also known as carbonized, carbon atoms.

In the example shown in FIG. 2, first wall 230 and second wall 232 collectively define a port 226 extending from second wall 232 to first wall 230. Port 226 provides access to an interior of first wall 230 through a first hole 227 formed in second wall 232 and a second hole 229 formed in first wall 230. One may use port 226 to access fluid within first wall 230 or to dispense medications within inner tube among other uses. The reader can see in FIG. 2 that brace member 206i is disposed proximate first hole 227 and second hole 229 to support port 226. Brace members 206ii-206v are also depicted proximate ports to support them. Of course, the brace members may be positioned not near a port to support the first and second walls.

### Mechanical Part in the Form of a Stent Embodiment Three

Turning attention to FIG. 3, a third example of a mechanical part in the form of a stent, mechanical part 301 in the form of a stent 300, will now be described. As can be seen in FIG. 3, stent 300 includes a first wall 330, a second wall 332, and a plurality of brace members 328.

With reference to FIG. 3, the reader can see that first wall 330 defines an inner tube 304 and second wall 332 defines an outer tube 302. In the FIG. 3 example, inner tube 304 and outer tube 302 are carbon nanotubes.

As can be seen in FIG. 3, inner tube 304 includes a flared terminal end 334 and outer tube 302 includes a flared terminal end 335. The flared terminal ends help to hold stent 300 in place by engaging surrounding tissue. Inner tube 304 and outer tube 302 include flared terminal ends on opposite longitudinal ends in the FIG. 3 example. In other examples, one or both terminal ends of the inner and outer tubes of the stent are not flared.

As can be seen in FIG. 3, plurality of brace members 328 is disposed between inner tube 304 and outer tube 302. Brace member 306i is a pyrolyzed carbon fullerene 307 forming a substantially spherical structure. Brace member 306i further includes a second carbon fullerene 312 contained within the substantially spherical structure of carbon fullerene 307. Second carbon fullerene 312 is not pyrolyzed, which facilitates it bonding to pyrolyzed carbon fullerene 307 at a desired position inside the substantially spherical structure of carbon fullerene 307.

In the present example, pyrolyzed carbon fullerene 307 comprises C₆₀ carbon and second carbon fullerene 312 comprises C₆₀ carbon. Second carbon fullerene 312 is chemically bonded to pyrolyzed carbon fullerene 307 within the substantially spherical structure of pyrolyzed carbon fullerene 307.

As can be seen in FIG. 3, brace member 306ii includes a pyrolyzed carbon fullerene 310 defining a substantially spherical structure. Unlike brace member 306i, pyrolyzed carbon fullerene 310 does not include an interior fullerene like second carbon fullerene 312 inside pyrolyzed carbon fullerene 307.

### Mechanical Part in the Form of a Stent Embodiment Four

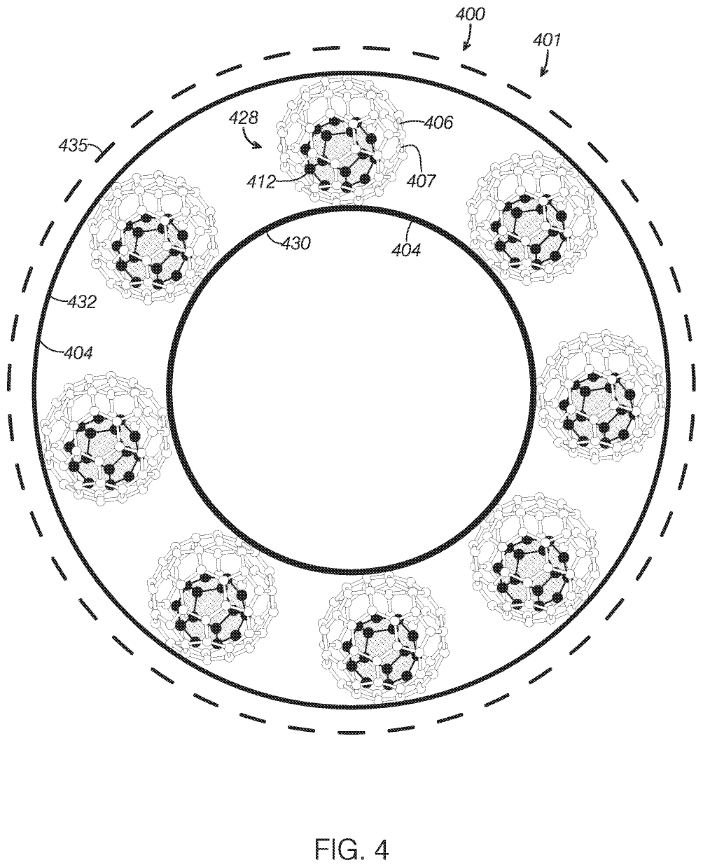

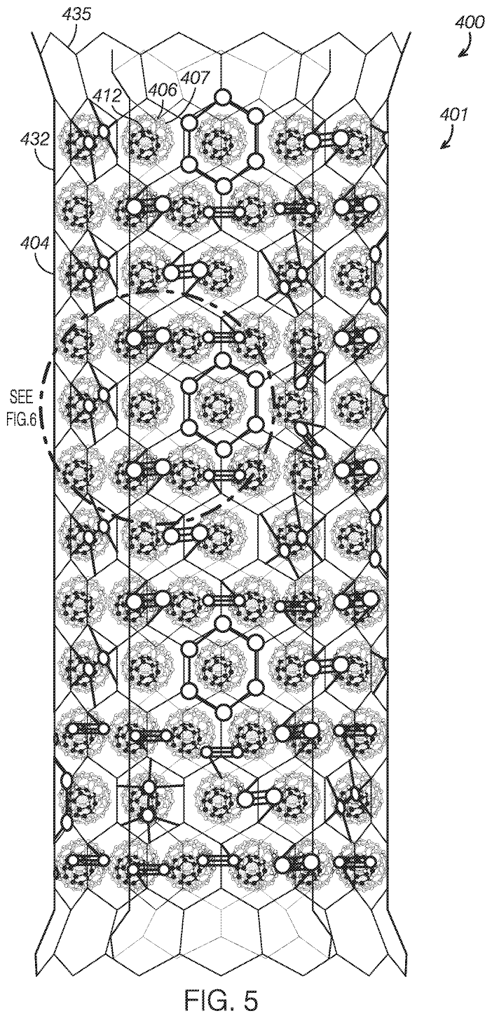

Turning attention to FIGS. 4-6, a fourth example of a mechanical part in the form of a stent, mechanical part 401 in the form of a stent 400, will now be described. As can be seen in FIGS. 4 and 5, stent 400 includes a first wall 430, a second wall 432, and a plurality of brace members 428.

With reference to FIGS. 4 and 5, the reader can see that first wall 430 defines an inner tube 404 and second wall 432 defines an outer tube 402. In the example shown in FIGS. 4-6, inner tube 404 and outer tube 402 are carbon nanotubes. As shown in FIGS. 4 and 5, outer tube 402 includes a flared terminal end 435 and a flared terminal end opposite terminal end 435.

As can be seen in FIGS. 3-6, the density of brace members 406 in plurality of brace members 428 is higher than the density of brace members 306 in plurality of brace members 328 depicted in FIG. 3. The increased density of brace members 406 increases the stiffness and rigidity of stent 400 relative to stent 300. The number of brace members may be selected to provide a desired amount of stiffness and rigidity.

In the example shown in FIGS. 4-6, each brace member 406 is a pyrolyzed carbon fullerene 407 forming a substantially spherical structure. Brace member 406 further includes a second carbon fullerene 412 contained within the substantially spherical structure of carbon fullerene 407. Second carbon fullerene 412 is not pyrolyzed, which facilitates it bonding to pyrolyzed carbon fullerene 407 at a desired position inside the substantially spherical structure of carbon fullerene 407. In the present example, pyrolyzed carbon fullerene 407 comprises C₆₀ carbon and second carbon fullerene 412 comprises C₆₀ carbon.

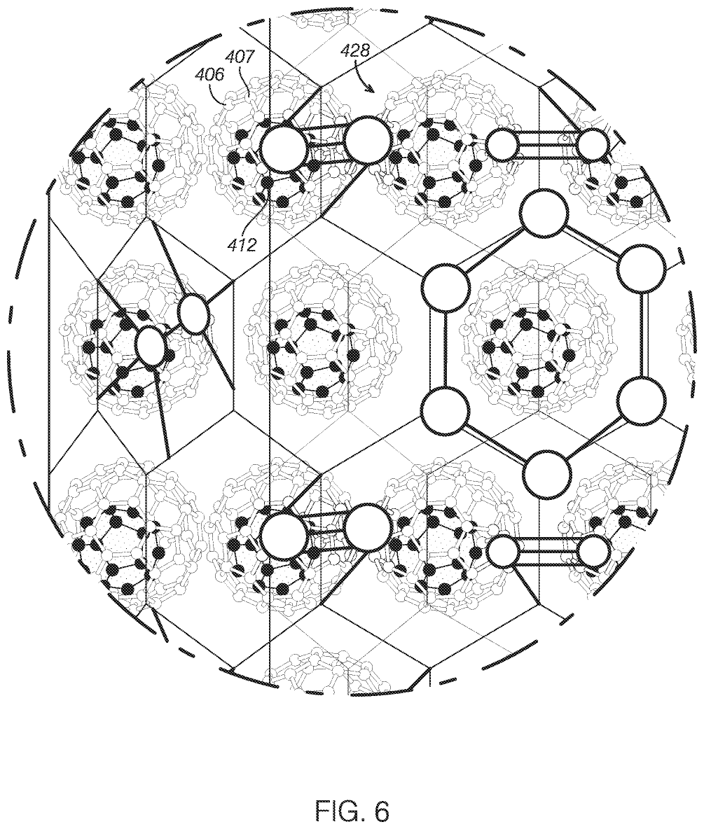

With reference to FIGS. 5 and 6, the reader can see how brace members 406 are arranged and chemically bonded within stent 400. The arrangement of brace members 406 and the different types of chemical bonds between them serves to form ports in stent 400 in desired positions and to provide structural integrity to stent 400. In other examples, the brace members are arranged and bonded to each other and to the inner and outer walls differently.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A stent comprising
an outer tube;
an inner tube coaxially aligned with the outer tube and contained within the outer tube; and
a brace member disposed between the inner tube and the outer tube, the brace member comprised of pyrolytic carbon.

2. The stent of claim 1, wherein the brace member includes carbon fullerene forming a substantially spherical structure.

3. The stent of claim 2, wherein:
the carbon fullerene defines a first carbon fullerene and is pyrolyzed; and
the brace member further includes a second carbon fullerene contained within the substantially spherical structure of the first carbon fullerene and the second carbon fullerene is not pyrolyzed.

4. The stent of claim 1, wherein the brace member includes pyrolytic carbon forming a sheet.

5. The stent of claim 1, wherein the brace member includes pyrolytic carbon forming a pyramid.

6. The stent of claim 1, wherein the brace member includes super pyrolytic carbon.

7. The stent of claim 1, wherein the brace member includes pyrolytic carbon interconnected with non-pyrolytic carbon in a zig-zag arrangement.

8. The stent of claim 1, wherein the inner tube and the outer tube are comprised of carbon nanotube.

9. The stent of claim 1, wherein the outer tube and the inner tube collectively define a port extending from the outer tube to the inner tube to provide access to an interior of inner tube.

10. The stent of claim 9, wherein the brace member is disposed proximate the port to provide structural stability for the port.

11. The stent of claim 1, further comprising a plurality of brace members, wherein the number of brace members is selected to provide a desired amount of stiffness between the outer tube and the inner tube.

12. A mechanical part comprising:
a brace member including:
a first carbon fullerene forming a substantially spherical structure, the first carbon fullerene being pyrolyzed; and
a second carbon fullerene contained within the substantially spherical structure of the first carbon fullerene.

13. The mechanical part of claim 12, wherein the first carbon fullerene comprises C₆₀ carbon.

14. The mechanical part of claim 13, wherein the second carbon fullerene comprises C₂₀ carbon.

15. The mechanical part of claim 12, wherein the second carbon fullerene is chemically bonded to the first carbon fullerene within the substantially spherical structure of the first carbon fullerene.

16. The mechanical part of claim 12, wherein the brace member is disposed between a first wall and a second wall.

17. The mechanical part of claim 16, wherein:
the second wall forms an outer tube; and
the first wall forms an inner tube and is contained within the outer tube.

18. The mechanical part of claim 17, wherein the inner tube and the outer tube are comprised of carbon nanotube.

19. The mechanical part of claim 17, wherein the inner tube, the outer tube, and the brace member collectively define a stent.

20. The mechanical part of claim 17, wherein the outer tube flares at a terminal end.
